(19) United States
(12) Patent Application Publication
(10) Pub. No.: US 2022/0079513 A1
(43) Pub. Date: Mar. 17, 2022

(54) HOUSEHOLD APPLIANCE ACTIVATION SYSTEM

(71) Applicant: VORWERK & CO. INTERHOLDING GMBH, Wuppertal (DE)

(72) Inventors: Andrej Mosebach, Bochum (DE); Christian Holz, Dortmund (DE)

(21) Appl. No.: 17/532,354

(22) Filed: Nov. 22, 2021

Related U.S. Application Data
(63) Continuation of application No. 16/263,917, filed on Jan. 31, 2019.

(30) Foreign Application Priority Data
Feb. 1, 2018 (EP) 18154688.8

(51) Int. Cl. A61B 5/00; A61B 5/11; A61B 5/0205; G16H 50/20; G16H 50/50; A61B 5/01

(52) U.S. Cl. CPC A61B 5/4809; A61B 2562/0247; A61B 5/4815; A61B 5/11; A61B 5/0008; A61B 5/02055; G16H 50/20; G16H 50/50; A61B 5/1116; A61B 5/4806; A61B 5/7264; A61B 5/01; A61B 2503/04; A61B 5/6831; A61B 2562/0271; A61B 2562/0261; A61B 2562/0219; A61B 5/4812

(57) ABSTRACT

A system includes a household appliance and a sensor. The sensor is configured to output a sensor signal to the household appliance. The household appliance is configured to be activated based on the sensor signal received from the sensor.

### PRIORITY CLAIM

This application is a continuation of co-pending U.S. Utility patent application Ser. No. 16/263,917, filed Jan. 31, 2019 that claims priority to European Application No. 18154688.8, filed Feb. 1, 2018, the disclosures of each of which are hereby incorporated in their entirety herein.

### FIELD OF THE DISCLOSURE

The present disclosure concerns a system comprising a sensor, which can measure a vital parameter of an organism and output a measurement signal corresponding thereto, and a household appliance which can be activated by an evaluation unit based on the measurement signal of the sensor.

### BACKGROUND

Vital parameters such as pulse frequency provide information about the current condition of an organism. There are systems which, for example, measure a vital parameter of an athlete by means of a sensor and which can evaluate and record the measurement signal by an evaluation unit. The athlete thus receives information about his physical condition within the recording period. The documents US2015/094544A1, US2012/253220A1 and US2016/287076A1 disclose similar monitoring systems for adult persons and babies.

It is object of the present disclosure to provide a further developed system which enables an enhanced functionality especially with regard to an interaction with other devices.

### SUMMARY

To solve the problem, a system, a method, and a computer program product according to the independent claims are provided. Advantageous embodiments result from the dependent claims.

A system for solving the problem comprises a sensor, which can measure a vital parameter of an organism and output a measurement signal corresponding thereto. The system comprises an evaluation unit, which can evaluate the measurement signal of the sensor. The evaluation unit can forecast a time of the occurrence of a change of the condition of the organism based on the evaluated measurement signal.

A vital parameter describes an organism (living being), and can be specified by a measured value. A vital parameter usually describes a basic function or vital function of an organism. The vital parameter, i.e. the measured value, can be determined using a sensor.

A measurement signal is in particular an analog signal, whose voltage, current strength and/or frequency correlates with the measured vital parameter, i.e. its measured value. An evaluated measurement signal normally results from a signal processing of the measurement signal, for example by means of an analog-to-digital conversion and/or a signal change by an algorithm. The evaluated measurement signal is preferably digital. In particular, the evaluated measurement signal can indicate the measured value of a vital parameter.

An evaluation unit preferably comprises a processor and a memory with a computer program code, i.e. instructions (commands) storable in the memory. The processor, the memory and the computer program code are configured in such a way that a method with several method steps can be carried out. A vital parameter can, for example, be determined through method steps based on a measurement signal. The expressions “based on” or “in dependency of” indicate an input variable for determination by the evaluation unit. In particular, the measurement signal and/or the evaluated measurement signal is such an input variable. Preferably, determining is carried out by an algorithm that is reflected in a computer program code and/or can be executed by method steps.

Change of condition means a specific change of condition, i.e. a change of condition (state) which is defined in the evaluation unit. For example, the change of condition is stored in the evaluation unit along with other changes of conditions, so that the change of condition of the organism can be assigned by the evaluation unit. In particular, the assignment to a change of condition can be made by assigning a specific sensor or several specific sensors to the specific change of condition. The evaluation of the evaluated measurement signal of this sensor or these sensors, which are assigned to a specific change of condition, is then used specifically for forecasting this specific change of condition. Preferably several changes of conditions can be forecasted based on the evaluated measurement signal of several sensors, wherein for each change of condition different evaluation algorithms and/or different combinations of the sensors are stored in the evaluation unit.

Change of condition of the organism means a change from a before-condition to an after-condition. Ideally, the before-condition is before the (point in) time and the after-condition is after the (point in) time of occurrence of a change of condition. A change of condition is, for example, a change from a sleep condition as a before-condition to a wake condition as a after-condition. A change of condition is for example a change from a closed bladder organ as before-condition to an open bladder organ as after-condition with escaping urine or a closed intestine as before-condition to an open intestine as after-condition with escaping bowel movement. A change of condition is, for example, a change from a non-crying condition as a before-condition to a crying condition as an after-condition, especially to signal hunger and/or thirst. But it can also be vice-verse. For example, a change of condition can be a change from a waking condition as before-condition to a sleep condition as after-condition.

A time for the occurrence of a change of condition of the organism is the point in time of the change from the before-condition to the after-condition. Since a change of condition itself usually takes a certain amount of time, e.g. the opening of the eyes when changing from a sleep condition to a waking condition, the (point in) time is to be understood as an approximate (point in) time, which ideally lies in the middle of the process of the change of condition. In particular, several points in time can define a time period, for example, if the process of changing the condition takes a long time.

Forecasting means predicting. The forecasting of a time of occurrence of a change of condition therefore takes place before the actual occurrence of the change of condition. In general, the process of change of condition has not yet started when the evaluation unit forecasts or outputs a forecasted time. Forecasting explicitly excludes the case in which a change of the condition is caused in a planned manner, like waking up at a previously defined time for example by an alarm clock. This point in time is then in an ordinary course not “forecasted” within the meaning of this disclosure, since the time of waking up is predefined and not linked to vital parameters.

The forecasting of a (point in) time of (for) the occurrence of a change of condition of the organism by the evaluation unit enables a device, in particular a household appliance, to carry out an action and/or take an operational state depending on the forecasted (point in) time.

If, for example, the organism is an infant, forecasting the time of waking up can activate a device for heating up a milk bottle or a kitchen appliance for preparing baby food so early that the preparation of the milk bottle or the baby food is completed shortly before or at least at the same time or approximately at the same time as the infant wakes up.

Alternatively or additionally, the device may be a notification device notifying a person of a forecasted change of condition and/or the forecasted time of the change of condition. In particular, a notification device may inform about a current condition and/or report a monitoring event related to a current condition. A notification device can, for example, be a wristband. In particular, the notification device may emit a sound, vibrate or otherwise alert a person and/or wake the person from sleep. In one embodiment, several persons who can be notified each carry a vital parameter sensor. The person who sleeps, based on the evaluated vital parameter sensor, least deeply or is currently in the REM sleep phase can then be woken up at night by vibration. The other person, for example, who is currently in a deeper sleep, can then continue to sleep.

In particular, the notification device has a display unit for displaying at least one piece of information of the notification. In one embodiment, the device is a smartphone or tablet PC. A person can thus be notified before a forecasted change of condition, so that a corresponding measure can be taken in good time before the change of condition occurs.

If, for example, the change of condition is the waking up of an infant and the forecasted time is the time of waking up, a parent (only one parent) can be woken and notified, particularly at night, in such a way that the parent has just enough time to bring a bottle of milk and/or baby food (gruel) in a ready-to-feed state to the infant before the infant wakes up. If the device for heating up the milk bottle or the kitchen appliance for preparing the baby food (gruel) has already been activated in dependency of the forecasted time of waking up, the parent only needs to bring the bottle or the baby food to the infant in order to feed it to the infant immediately after waking up. The sleeping time of the parents can thereby be maximized.

By forecasting a time of the occurrence of a change of condition, time can be saved, rest time be increased and a particularly high degree of automation be achieved. Further examples are described below.

The device can be a front door lock, a motorized roller shutter and/or a smart home server. The forecasted time of a change of condition can be the time of falling asleep. The device can therefore carry out an action or take an operating state before the time of falling asleep. For example, the front door lock can be automatically locked, roller shutters and/or windows can be automatically closed at a defined time offset before the forecasted time of falling asleep. The forecasted time of a change of condition can be the waking up time. For example, the heating, especially in the bathroom, can then be activated at a defined time offset before the forecasted time of waking up, so that a person enjoys a preheated bathroom after waking up.

The organism can be an animal or a pet. The owner of the animal or pet can be notified before the animal wakes up, for example, to ensure that all doors and windows are closed. Injury to a cat from a tilted window can be avoided, for example.

In one embodiment, a urine and/or stool container for a person in need of care can be brought into operating position in good time before urine or bowel movement.

If the device is the smart home server, the room temperature and/or the lighting can be changed depending on the forecasted time, in particular the time of falling asleep and/or waking up. This enables the temperature or light intensity to be lowered evenly over a period before falling asleep. The temperature or light intensity can be raised during a period before waking up.

It can also be made possible for a person to take freshly baked rolls and/or fresh coffee from a corresponding kitchen device such as an oven, coffee machine or a kitchen appliance after waking up.

In one embodiment, the evaluation unit is equipped with a machine learning algorithm in order to forecast the time of occurrence of the change of condition of the organism based on the evaluated measurement signals.

A machine learning algorithm generally assigns an output variable to one or more input variables and usually outputs it. A machine learning algorithm is often the basis for so-called “artificial intelligence”, wherein the machine learning algorithm “learns” from experience and “recognizes” patterns and laws self-sufficiently even in unknown data. A machine learning algorithm can be formed by a so-called “neural network” or it can comprise this in the form of a corresponding program. In particular, a machine learning algorithm is generated by a modelling phase and a subsequent identification phase, in order to finally be able to forecast a time for the occurrence of a change of condition in a forecast phase. In particular, the modelling phase takes place at the manufacturer. The identification phase can take place at the manufacturer and/or at the end user. The forecast phase is then always carried out at the end user. For testing purposes, the forecast phase can be carried out at the manufacturer.

In the modelling phase, a mathematical model, i.e. equation system, is created to assign one or more input variables to an output variable. A correlation of one or more vital parameters with a change of condition is taken into account, i.e. reflected in the mathematical equation system. Preferably in the modelling phase, a dynamic model and/or differential equation system is provided for the forecast of the time of the occurrence of a change of condition of an organism on the basis of the evaluated measurement signal. In the model or the differential equation system, the evaluated measurement signals of a predefined sensor or several predefined sensors serve as input variable or input variables and the forecasted time of occurrence of a certain change of condition as the output variable.

In the identification phase, the machine learning algorithm is supplied with a plurality of value pairs each with an input variable and an output variable or with several input variables and one output variable, respectively. In this way, the machine learning algorithm is optimized and adapted to reality. In particular, constants are optimized in a differential equation system of the machine learning algorithm based on the supplied value pairs. By providing a feedback unit, an output variable can be supplied to the machine learning algorithm by the end user, which will be discussed in more detail later.

In the forecast phase, the machine learning algorithm is used to forecast the time of occurrence of the change of the condition of the organism based on the evaluated measurement signals.

By equipping the evaluation unit with the machine learning algorithm, a particularly precise forecast of the time of the occurrence of a change of condition can be enabled. It is possible to generalize and derive regularities of the course of one or more vital parameters with regard to the change of condition. In this way, a forecast that takes the peculiarity of the organism into account can be obtained.

In one embodiment, the machine learning algorithm, which can include an extrapolation parameter and/or a threshold, is used for the entire forecast, so that the output variable that is output by the machine learning algorithm corresponds to the time of the occurrence of the change of condition. In an alternative or additional embodiment, the machine learning algorithm outputs only an intermediate variable, such as an extrapolation parameter and/or a threshold, for determining the forecasted time of the occurrence of the change of condition.

In one embodiment, the evaluation unit extrapolates a time course of the evaluated measurement signals. Information relevant for the forecast of the time of the change of condition can thus be determined particularly easily. The time course of the evaluated measurement signals can be graphically illustrated as a measurement curve, e.g. in FIGS. 3 and 4, in which the values of the evaluated measurement signals are plotted over the time axis. As in FIGS. 3 and 4, the extrapolation of the time course of the evaluated measurement signals can be represented as an extrapolated curve that follows and continues the measurement curve in the direction of the time axis. In particular, the extrapolation is based on at least one extrapolation parameter that influences the course of the extrapolated curve. Preferably, for the extrapolation, a curve function is stored that includes at least one extrapolation parameter. In one embodiment, the curve function is part of the machine learning algorithm.

In one embodiment, the evaluation unit comprises a threshold for the evaluated measurement signals for indicating a change of condition. Threshold means threshold value. Indicating of a change of condition means that if an evaluated measurement signal has reached or exceeded the threshold or the threshold value, this is an indication of the occurrence of the change of condition. By providing a threshold, the time for occurrence of a change of condition can be implemented particularly easily. In one embodiment, the threshold is part of the machine learning algorithm.

In one embodiment, the evaluation unit identifies the time as the forecasted time, at which the extrapolated time course of the evaluated measurement signals reaches the threshold. The evaluation unit thus evaluates the forecasted time of the occurrence of the change of condition by a point of intersection of the extrapolated curve with the threshold, as exemplarily shown in FIGS. 3 and 4. The time of occurrence of the change of condition can thus be forecasted using a particularly simple algorithm. In one embodiment, this algorithm is part of the machine learning algorithm.

In one embodiment, two thresholds are provided, in particular a minimum threshold or a minimum threshold value and a maximum threshold or a maximum threshold value. A forecasted earliest (point in) time then results from the intersection of the extrapolated curve with the minimum threshold value and a latest (point in) time from the intersection of the extrapolated curve with the maximum threshold value. The probability of the occurrence of the change of condition before or after the forecasted (point in) time can thus be quantified, for example in the form of a confidence level.

In one embodiment, the threshold and/or the extrapolated time course of the evaluated measurement signals are determined by the machine learning algorithm. The threshold or the extrapolation of the time course of the evaluated measurement signals can thus be determined particularly precisely, even if several input variables are processed.

In one embodiment, an input interface for a user is provided. User means end user like e.g. a parent of an infant. The user can be the organism itself, if the system is connected to the Smart Home server for example to increase the living comfort. An input interface allows in particular inputting of a number or a text. Preferably an input interface has a display to show the inputs. In one embodiment, the input interface is a smartphone or tablet PC. In one embodiment, an app for a smartphone serves as an input interface.

In one embodiment, the user can set the threshold, in particular via the input interface. The threshold can thus be adapted to the user's experience. Sometimes parents perceive a correlation between vital parameters and a time of a change of condition such as the time of waking up and can thus adjust the threshold in a targeted way, e.g. a threshold for body temperature. The user is thereby given the opportunity to incorporate his own empirical values into the forecast of the time of occurrence of a change of condition and to enable a particularly reliable forecast adapted to the organism.

In one embodiment, the user can set a sensitivity or confidence level of the system for the forecast of the time via the input interface. The higher the sensitivity or confidence level is set, the earlier is the time at which the change of condition is forecasted to occur. In this way, it can be avoided that the forecasted (point in) time lies behind the actual (point in) time. In addition to that, the system can be adapted to customer requirements.

In one embodiment, a feedback unit is provided by means of which a user can give a feedback about the occurrence of a change of condition to the machine learning algorithm. By providing a feedback unit, an output variable can be fed to the machine learning algorithm by the end user. The machine learning algorithm can thus, for example, “learn” typical waking up times of a certain infant and take them into account. Preferably the feedback unit includes a button. A particularly simple and uncomplicated way of giving feedback by the user can thus be made possible. When the infant wakes up, the button can be pressed, for example, to inform the system about the time of waking up in a precise and reliable manner. Preferably, only one push-button is provided as the entire user interface of the feedback unit. In an alternative or additional embodiment, the input interface can be used as a feedback unit. A particularly detailed feedback for the machine learning algorithm can thus be enabled.

In one embodiment, a sensor is connected to the feedback unit or represents a feedback unit, in particular a humidity sensor. A change of condition, e.g. urine leakage or sweat discharge, can thus be automatically provided to the machine learning algorithm as an output variable for its optimization.

In one embodiment the sensor is a gyrometer. For example, a gyrometer is used to measure a rotary movement. By measuring the rotary motion, a measure of the activity can be determined to describe the organism, which can be correlated with a change of condition, e.g. waking up. In particular, a change of direction of a rotary movement is recorded and/or measured per time interval of e.g. ten seconds. If, for example, at least six changes of direction take place in a ten-second period, this is an indication of an infant's waking condition. At the same time, there is a steep increase in the number of changes of direction within a period of, for example, ten minutes before waking up, with a particularly approximately constant gradient over time. The use of a gyrometer as a sensor thus enables a particularly reliable forecast of the time of waking up of an infant.

Preferably, the gyrometer is attached to a wrist or ankle of an infant, particularly by means of a bracelet or foot band. In particular, the gyrometer is used to determine acceleration.

Alternatively or additionally, the sensor is a force sensor, a force transducer, a piezo sensor and/or a strain gauge.

In an alternative or complementary embodiment, the sensor or a further sensor is a skin contact sensor for measuring body temperature. The body temperature is a vital parameter that correlates, among others, with the sleep/wake cycle. A change of condition, such as waking up, can thus be forecasted particularly easily.

In particular, the at least one sensor can be a humidity sensor for detecting a wet diaper and/or sweat discharge, a movement sensor mat for activity measurement, an odor sensor, in particular for methane, a pulse meter, a blood pressure meter, a brain current sensor for EEG and/or ECG, an oxygen measurement sensor, in particular for determining the sleeping section, an MRI device particularly for determining a wake-up time, a thermal imaging camera, a night vision camera particularly for detecting characteristic movement sequences, a camera with color resolution particularly for assigning the skin color, a blood sugar level sensor, a CO₂ measuring device for breathing air, a pupil size measuring device, a blinking frequency measuring device particularly for forecasting a time at which people fall asleep, and/or a breathing frequency measuring device. The skin contact sensor can be used alternatively or additionally to measure electrical voltage fluctuations on the skin surface.

In one embodiment, at least two sensors are provided for different vital parameters. The time of occurrence of a change of condition can thus be forecasted particularly reliably by taking two different vital parameters into account. For example, the two different vital parameters are the activity measured by the gyrometer and the body temperature measured by the skin contact sensor. The body temperature indicates whether the organism is, for example, in the middle or at the end of the sleep phase. The activity indicates a REM phase or a soon waking up. By linking these two vital parameters, the body temperature can be used, for example, to roughly identify the sleeping section and the activity can be used to concretize the remaining time until waking up. If, for example, an increased activity is measured in a middle sleeping section, it can be either a REM phase or a nightly waking up at night, but not the morning waking up.

In one embodiment, a combination of gyrometer and strain gauge can be used to measure activity, pulse and/or respiratory rate and thus different vital parameters.

In particular, the sensor or sensors are attached to a sleeping place of the organism, in or on a blanket and/or in or on a sleeping bag.

In one alternative or complementary embodiment, the forecasted time of the occurrence of the change of condition can be carried out based on evaluating of at least two different vital parameters. A particularly reliable forecast is thus enabled. For example, the care needs of an infant or a person in need of care can be determined at an early stage, so that care can be provided particularly quickly and promptly.

In particular, the vital parameters may be two or more of the following: body temperature, activity, pulse, blood oxygen content, blood sugar level, brain current, characteristic movement patterns, CO₂ respiratory air content, respiratory rate, pupil size and/or blinking frequency.

In one embodiment, an environmental information can be taken into account in order to determine the forecasted time of the occurrence of the change of condition. The evaluation unit and/or the machine learning algorithm are configured accordingly. In particular, the environmental information is the weather or a weather forecast or moon phase calendar. If, for example, a thunderstorm is forecasted, the probability of a faster waking up increases, which can be taken into account in the forecast. In particular, the environmental information includes a timetable for a bus, a train, a garbage collection service and/or a robot vacuum cleaner.

Preferably, the evaluation unit has an internet interface to connect to a weather service, a smart home server, for example with the schedule of the robot vacuum cleaner, and/or a public timetable information for buses, trains and garbage collection service.

In one embodiment, a temperature sensor for recording the room temperature, a brightness sensor for recording the room brightness, a humidity sensor for recording the room humidity and/or a microphone for recording traffic noise, ambient noises or personal noises such as talking or snoring are provided.

In one embodiment, the system comprises a household appliance that can be connected to the evaluation unit for exchanging data in order to carry out an action in dependency of the forecasted time. Thus, an action is programmed in the household appliance which is executed by the household appliance when the household appliance receives a forecasted time from the evaluation unit, wherein the forecasted time is taken into account in the execution of the action. The user can thereby save time, increase rest time and achieve a very high degree of automation as shown by the examples of such actions described above.

Household appliance means an electrically operated appliance for use in private households. This may be an electric kitchen appliance or a cleaning appliance with an interface to the internet, a WLAN interface and/or a connection to a smart home server. A household appliance within the meaning of this disclosure also includes do-it-yourself appliances such as cordless screwdrivers or drills as well as garden appliances such as lawn mower robots. A cleaning appliance is, for example, a robot vacuum cleaner. A household appliance or device can also be a smart home server that can automate processes with the help of networked and remote-controlled devices, switches and sensors, thus enabling particularly high living quality, safety and energy efficiency. Preferably, the smart home server is connected to home installations, building technological infrastructure and house devices such as lamps, blinds, shutters, doors, windows, heating, oven, stove, kitchen machine, refrigerator, washing machine, vacuum cleaner, television and/or audio equipment. In particular, the household appliance has a WLAN interface and/or an Internet interface.

A further aspect of the present disclosure concerns the use of the system according to the above described aspect of the present disclosure, wherein the forecasted time of the occurrence of the change of condition of the organism is the wake-up time of an infant. By using the system in this way, a particularly reliable forecast and a particularly high time saving and rest period for the parents can be achieved.

A further aspect of the present disclosure concerns a system, in particular according to the aspect of the disclosure described above, comprising a sensor which can measure a vital parameter of an organism and output a corresponding measurement signal. The system comprises an evaluation unit that can evaluate the measurement signal of the sensor. The evaluation unit can use the evaluated measurement signal to detect the occurrence of a defined change of condition. A person can thus be informed promptly about the occurrence of the change of condition. Particularly in combination with the forecast, additional functions such as emergency call messages can be covered by the system.

A further aspect of the disclosure concerns a method in which a vital parameter of an organism is measured with a sensor and a measurement signal corresponding thereto is output. An evaluation unit evaluates the measurement signal of the sensor and forecasts a time of occurrence of a change of condition of the organism based on the evaluated measurement signal. The features, embodiments and effects of the system for solving the problem described at the beginning also refer to this method accordingly.

A further aspect of the disclosure concerns a computer program product. The computer program product includes instructions (commands), which when executing the program of the computer program product by a computer, cause the computer to perform the steps of the method according to the preceding aspect of the disclosure. In particular, the computer is the evaluation unit. The features, embodiments and effects of the system for solving the problem described at the beginning also refer to this computer program product accordingly.

In the following, embodiment examples of the disclosure are explained in more detail also using figures. Features of the embodiment examples and further alternative or supplementary embodiment described below can be combined individually or in a plurality with the claimed objects. The claimed scope of protection is not limited to the embodiment examples.

### BRIEF DESCRIPTIONS OF THE DRAWINGS

It is shown:

FIG. 1: Schematic illustration of a system for forecasting a time of waking up of an infant;

FIG. 2: Schematic illustration of a system with several sensors, an evaluation unit and notification devices;

FIG. 3: Schematic illustration of a diagram with a measurement curve of the activity, an extrapolated curve, a threshold and a forecasted time of the occurrence of a change of condition from sleep condition to waking up condition;

FIG. 4: Schematic illustration of a diagram with a measurement curve of the body temperature, an extrapolated curve, a threshold and a forecasted time of the occurrence of a change of condition from sleep condition to waking up condition.

### DETAILED DESCRIPTION

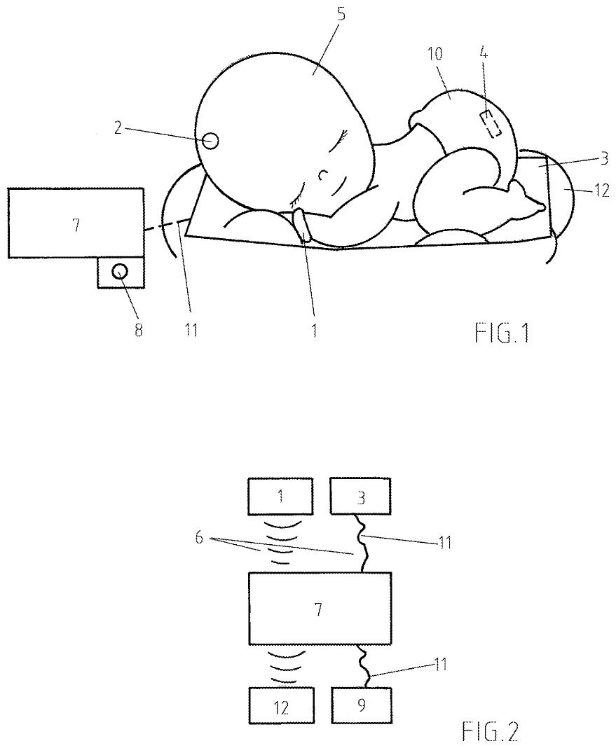

FIG. 1 shows an infant as the organism 5 in sleep condition. The infant has a gyrometer 1 on his wrist, particularly integrated into a bracelet. The infant has a skin contact sensor 2 which is preferably attached to the head, particularly preferred at the forehead or temple, with contact to the skin surface. In particular, the skin contact sensor 2 is attached with a plaster or is held by a headband and pressed against the skin surface. The infant lies on a movement sensor mat 3 which is equipped with force sensors such as strain gauges to measure the movement of the infant. The movement sensor mat 3 is arranged on a bed 12 in which the infant lies. A humidity sensor 4 is mounted in a diaper 10 of the infant to measure or detect leaking urine. An evaluation unit 7 can optionally be connected via a cable 11 to one of the sensors (1 to 4), preferably to the movement sensor mat 3.

The evaluation unit 7 is connected to a feedback unit 8, which can be operated by means of a head (button). If, for example, the time of waking up is forecasted, the button can be pressed during the actual waking up of the infant to provide the system with feedback on the actual time of waking up. The system can thus optimize the stored algorithms, in particular a machine learning algorithm, for forecasting the time of waking up.

The sensors 1, 2 and 4 are preferably wirelessly connected to the evaluation unit 7 for exchanging of data.

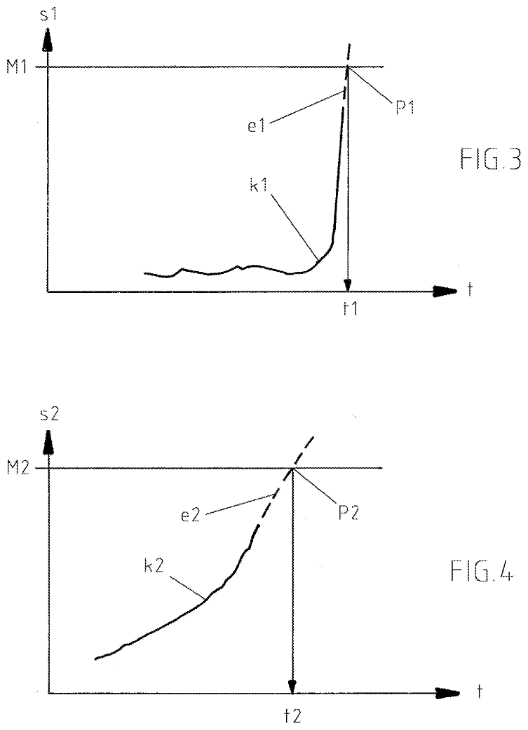

FIG. 2 shows the schematic structure of the system, in particular the system of FIG. 1. A gyrometer 1 transmits its measurement signal 6 to the evaluation unit 7 wirelessly, in particular via radio connection. A movement sensor mat 3 transmits its measurement signal 6 to the evaluation unit 7 via a cable. The optionally provided but not shown feedback unit 8 is connected to the evaluation unit 7 either via a cable or wirelessly for exchanging data. The evaluation unit 7 processes the measurement signals 6 in order to obtain one or more evaluated measurement signals k1, k2, which indicate one or more vital parameters or at least correlating characteristic values. By using an algorithm, the evaluation unit 7 determines a forecasted time t1, t2 of occurrence of a change of condition. For example, this determination can be conducted as described below with reference to FIGS. 3 and 4. If a (point in) time is forecasted or if a forecasted time falls within a previously defined time offset (time distance/interval before) to the forecasted time of the change of condition, e.g. ten minutes, the forecasted time is transmitted to a notification device 12 and/or a household appliance 9. In particular, information about the change of condition is transmitted together with the forecasted time. These notifications to the notification device 12 or the household appliance 9 can also be sent wirelessly or by cable as shown exemplarily in FIG. 2. In particular, the evaluation unit 7, the notification device 12 and/or the household appliance 9 have a Bluetooth interface and/or a WLAN interface in order to be able to transmit data wirelessly.

FIG. 3 shows a diagram of the activity s1, plotted over the time t, measured by the gyrometer 1. As an example, the forecast of a time of waking up is illustrated in FIG. 3, particularly the time of waking up of the infant of FIG. 1. The evaluated measurement signals form a measurement curve k1, which is represented by a solid line. The algorithm stored in the evaluation unit 7, in particular a machine learning algorithm, is able to extend the curve progression, based on the course of the measurement curve, into the future in form of the extrapolated curve e1. A threshold M1 is provided, which indicates the change of condition from the sleep condition to the waking up condition, abbreviated called time of waking up. The time t1 of the change of condition from the sleep condition to the waking up condition thus results from the intersection P1 of the extrapolated curve e1 with the threshold M1. This time t1 is forecasted on the basis of the course of the measurement curve and thus based on the measured movements.

FIG. 4 illustrates a further example for forecasting, for example, a time of waking up, in particular the time of waking up of the infant in FIG. 1. The diagram shows the body temperature s2, which is plotted over time t, measured and signal processed by the skin contact sensor 2. The evaluated measurement signals form a measurement curve k2, which is represented by a solid line. The algorithm stored in evaluation unit 7, in particular a machine learning algorithm, is able to extrapolate the curve progression into the future in form of the extrapolated curve e2 based on the course of the measurement curve. A threshold M2 is provided, which indicates the time of waking up. The forecasted time t2 results from the intersection P2 of the extrapolated curve e2 with the threshold M2. The time t2 of waking up can again be forecasted by temperature measurements.

In one embodiment, a consolidation algorithm is provided, which determines a consolidated forecasted time from both forecasted times t1 and t2, if the (point in) times t1 and t2 differ from each other. In particular, the consolidation algorithm is part of the machine learning algorithm.

In one embodiment, the course of the evaluated measurement signals k1 of the gyrometer 1 is only extrapolated, if the body temperature measured by the skin contact sensor 2 has reached a certain minimum temperature. Computer capacity can thus be saved. Furthermore, a (point in) time is extrapolated only when a soon waking up can be expected to be sufficiently probable based on the measured body temperature.

In one embodiment, the extrapolation of a course of evaluated measurement signals is only carried out if a predefined course scheme or a predefined minimum signal value has been reached. Computer capacity can thus be saved.

In one embodiment, an extrapolated time is sent to a device connected to evaluation unit 7, in particular household appliance 9 or notification device 12, not before a predefined time offset (time distance/interval) to the extrapolated time is reached or if the forecasted time falls within the time offset (time distance/interval). Preferably, the predefined time offset is five to fifteen minutes, for example ten minutes.

In one embodiment, the skin contact sensor 2 is arranged to measure the electrical voltage fluctuations on the skin surface. Measurement signals for an electroencephalogram can thus be measured. During the change of condition from sleep condition to waking up condition, a measured frequency of the voltage fluctuations changes from alpha waves to beta waves. Conversely, a measured frequency of the voltage fluctuations during the change of condition from waking up condition to sleep condition changes from beta waves to alpha waves. Alpha waves refer to a frequency range between 8 and 13 Hz. Beta waves refer to a frequency range between >13 and 30 Hz. The threshold is 13 Hz.

In particular, in the event of a recognized danger to life, the system may preferably contact an ambulance service via a router connected to the system and/or send relevant measurement data to the ambulance service for first aid support.

In one embodiment, an app for a smartphone is used to display the measurement signals 6, the measurement curve k1, k2, the extrapolated curve e1, e2, the threshold M1, M2 and/or the forecasted time t1, t2. This allows the user to perform his own analyses of the historical data.

As described above, the evaluation unit 7 is in one embodiment configured in such a way that the forecasted time t1, t2 is transmitted with (sent in) a defined time offset (time distance) before the forecasted time t1, t2 to a household appliance 9 to carry out an action.

In one embodiment, the household appliance 9 is an electric kitchen appliance or a cleaning appliance with an interface to the Internet, so that the evaluation unit 7 can be connected to the household appliance 9 for exchanging data. In one embodiment, the evaluation unit 7 only sends the extrapolated time to the household appliance 9 when the predefined time offset (time distance) to the extrapolated time t1, t2 has been reached. Preferably, the household appliance 9 then carries out the action in dependency of the forecasted time t1, t2. In one embodiment, the kitchen appliance is a food processor that preferably automatically prepares food, and/or the cleaning appliance is a robot vacuum cleaner for which a cleaning plan has been defined and stored.

The forecasted time equals to the extrapolated time. The defined time offset equals to the predefined time offset. A kitchen appliance has at least three functions: heating, crushing and mixing a food. In one embodiment, the forecasted time is transmitted immediately to the household appliance if the remaining time until the forecasted time is less than the defined or predefined time offset.

The action may be an immediate deactivation of the robot vacuum cleaner or the immediate start of automatic food preparation by the kitchen appliance, in particular the food processor.

In one embodiment, the evaluation unit 7 sends the extrapolated time t1, t2 to the kitchen appliance or the cleaning appliance as soon as the extrapolated time t1, t2 has been determined by the evaluation unit 7. The kitchen appliance or cleaning device can then be configured in such a way that the action is carried out not before the defined time offset to (being subtracted from) the forecast time t1, t2. Higher efficiency can thus be achieved because, for example, it allows the robot vacuum cleaner to first of all continue cleaning and the food processor not to start preparing the food until the time of the extrapolated time minus the defined time offset has been reached. In the variant of the robot vacuum cleaner, for example, the defined time offset corresponds to the time which the robot vacuum cleaner needs to return to the charging station. In the variant of the kitchen machine, the time offset can be defined automatically based on the food previously selected or preset by the user or it can correspond to its preparation time. If, for example, the food is a baby bottle of milk, the defined time offset corresponds to the preparation time for the milk plus a cooling phase. By means of this concrete arrangements described above, the resting time of the parents can be increased.

In one embodiment, more than one person are each equipped with a notification device 12. In particular, the notification device 12 measures vital data, i.e. at least one vital parameter, of the respective person by a sensor and transmits this to the evaluation unit 7. In particular, this is carried out in analogy to the vital parameter measurement as described above. Preferably, the measured vital data is evaluated by the evaluation unit 7, preferably in analogy to the example of a baby as described above. In this way, the evaluation unit 7 can determine the sleep state and/or rest state of the respective persons. In particular, the persons are persons for the care of an organism, e.g. the parents of a baby and/or other supervisors for the baby.

Preferably, the notification device 12 comprises a display and/or a unit for alerting the person, in particular a vibration transmitter. For example, a vibration transmitter allows only one parent to be woken by the alert, while the spouse sleeping next to him/her is not woken.

In one embodiment, the evaluation unit 7 is configured such that at least one person of the plurality of persons can be selected based on the measured vital parameters and only the at least one selected person can be alerted in a defined time offset to (before) the forecasted time or when a forecasted time of occurrence of a change of condition of the organism is determined. In particular, all persons with a notification device 12 whose measured vital parameters reach a threshold value or exceed a threshold value will be selected. The lower the sleep state or rest state of a person, the lower the value of the vital parameter.

Preferably, the threshold is defined in a way that when the threshold is reached or exceeded—compared to the state with the vital parameter below the threshold—the person is suitable for selection because he or she is easier to wake up, sleeps less deeply and/or suffers less stress from being alerted by the notification device. If the values of all people's vital parameters are below the threshold, either all people can be alerted or only the person whose vital parameters are closest to the threshold can be alerted.

If the evaluation based on the measured vital parameters shows that several persons are suitable for an alarm, all these several persons of the plurality of persons can thus be selected and alerted. If always all selected persons are alerted, the risk is reduced that no alerted person has taken notice of the alert.

In one embodiment, the notification device 12 and/or the evaluation unit 7 can establish and/or control a data connection to a kitchen appliance or a robot vacuum cleaner. A person who has just been alerted of a forecasted change of condition can then, without any loss of time, e.g. from his bed, use the notification device to control the kitchen appliance to prepare a meal or the robot vacuum cleaner to change a cleaning plan. In particular, to control includes to activating a predefined action, such as deactivating the robot vacuum cleaner or to triggering automatic food preparation by the kitchen appliance.

The variant with the kitchen appliance thus enables the alerted person to remain in bed for even longer because it was possible to program the kitchen appliance easily from the bed using the notification device 12. Here, programming means selecting the food and/or selecting the completion time for the food by entering or selecting a time offset before or after the forecasted time. These few minutes of time saved in bed, however, ensure a significantly lower loss of rest when the person gets up, because this gives the circulation, the body and the head sufficient opportunity to slowly move from the resting state to the active state.

In particular, the display of the notification device 12 shows information about the current status of the food preparation conducted by the kitchen appliance, the recipe currently being conducted and/or a control command from the evaluation unit to the kitchen appliance. In this way, the loss of rest can be further reduced. Preferably, a recipe or a dish can be selected for automatic preparation using the notification device 12. Preferably, the recipes or meals with the planned time period for preparation are then displayed. In particular, the user can enter the defined time offset via the notification device.

In particular, a cleaning plan for the robot vacuum cleaner is provided. Preferably, the cleaning plan includes a driving route and/or a cleaning schedule. For example, the schedule provides for the robot vacuum cleaner to regularly drive the route to clean the floor of living areas.

In the variant with the robot vacuum cleaner and its data connection and/or control by the notification device 12, it is possible for the alerted person to change the cleaning plan promptly and on the spot based on the event of which the person was informed as a result of the alert. If, for example, a baby wakes up at night, the cleaning plan can be changed immediately by the notification device 12 so that the robot vacuum cleaner is deactivated before the forecasted time, the robot vacuum cleaner does not drive to the child's room (change of the driving route) or the robot vacuum cleaner drives the route with a time delay (change of the cleaning schedule). This is in the case of babies of essential benefit for the following reasons. Because robot vacuum cleaners are dangerous for babies being located on the floor and because the operating noises usually do not wake up sleeping babies, vacuum cleaner robots are usually programmed to clean at night. However, a waking baby is put into a higher state of activity by the sounds and/or the sight of a moving robot vacuum cleaner, which puts an actually tired baby into a waking state, from which the baby can be brought back to sleep much more difficult and with more work. This can be prevented by the embodiment described above, so that the alerted person does not have to get up immediately to deactivate the robot vacuum cleaner before the forecasted time is reached. Instead, the person can stay in bed until the circulation, the body and the head have adapted to the forthcoming getting up, for example at night because of the waking up baby. The loss of rest can thus be significantly reduced by this embodiment.

In particular, information about the current status of the robot vacuum cleaner, the cleaning plan and/or a control command from the evaluation unit to the robot vacuum cleaner is shown on the display of the notification device 12. The loss of rest can thus be further reduced.

A kitchen appliance has at least the three functions of heating, chopping and blending a food. Preferably, the kitchen appliance can access stored recipes for a variety of foods. Preferably, a recipe can be displayed on the kitchen appliance via an interactive display, e.g. touch screen display, and processed by the user step by step. In one embodiment, the kitchen appliance can process a recipe completely self-acting and thus automatically prepare a food.

1. A kitchen appliance system comprising:
a kitchen appliance adapted for electrical activation by an evaluation unit; the kitchen appliance having at least the three functions of heating, chopping and blending a food, and having access to stored recipes for a variety of foods, which can be displayed on the kitchen appliance via an interactive touch screen display such that a user can process a recipe step by step, wherein the kitchen appliance is configured to allow processing a recipe completely self-acting to automatically prepare a food by means of heating, chopping and blending; and
a sensor configured to output a sensor signal indicating a vital parameter of an organism,
the evaluation unit including a processor and a memory storing instructions to be executed by the processor, the processor configured to
receive the sensor signals and evaluate the received sensor signals,
determine time information related to the vital parameter of the organism and a food that needs to be prepared by extrapolating a time course of the evaluated sensor signals and comparing the extrapolated time course with a predefined threshold for the extrapolated time course,
compare the time information of the evaluated sensor signals to a current time, wherein the time information corresponds to a time when the user needs the prepared food at the kitchen appliance, and
transmit an activation signal to the kitchen appliance to initiate an appliance operation based on a difference between the time information and the current time such that the food preparation is completed at the time when the prepared food is needed at the kitchen appliance, thereby saving the user a time to completion of the appliance operation.

2. The kitchen appliance system of claim 1, wherein the difference is a time period remaining between the current time and the time when the prepared food is needed at the kitchen appliance.

3. The kitchen appliance system of claim 2, wherein the processor transmits the activation signal only when the difference is less than or equal to a predefined time offset.

4. The kitchen appliance system of claim 3, wherein the time offset corresponds to the time period necessary to complete the appliance operation to prepare the food.

5. The kitchen appliance system of claim 4, wherein the time threshold is automatically defined based on a selection of the food by the user.

6. The kitchen appliance system of claim 5, wherein the food is baked goods.

7. The kitchen appliance system of claim 5, wherein the food is baby food.

8. The kitchen appliance system of claim 4, wherein the time offset is less than fifteen minutes.

9. The kitchen appliance system of claim 1, wherein the system is configured such that a user set the threshold via the touch screen display interface.

10. The kitchen appliance system of claim 9, wherein the sensor is a skin contact sensor for measuring body temperature and the vital parameter is the body temperature.

11. The kitchen appliance system of claim 10, wherein the system is configured such that the user wears a notification device comprising at least one of a display and a vibration transmitter for alerting the user based on the measured and evaluated vital parameter so that the user arrives at the kitchen appliance when the food is needed and the preparation is completed.

12. A household appliance system comprising:
a household appliance adapted for electrical activation by an evaluation unit; and
a sensor configured to output a sensor signal indicating a vital parameter of an organism,
the evaluation unit including a processor and a memory storing instructions to be executed by the processor, the processor configured to
receive the sensor signals and evaluate the received sensor signals,
determine time information related to the vital parameter of the organism by extrapolating a time course of the evaluated sensor signals,
compare the time information of the extrapolated time course of the evaluated sensor signals to a current time, wherein the time information corresponds to a time of arrival of a user at the household appliance, and
based on a difference between the time information of the extrapolated time course and the current time, transmit an activation signal to the household appliance to initiate an appliance operation such that the appliance operation is completed prior to the time of arrival of the user thereby saving the user a time to completion of the appliance operation.

13. The household appliance of claim 12, wherein the time threshold corresponds to a time at which the appliance operation will be completed, and wherein the difference is a time period remaining between the time of arrival of the user at the household appliance and the time at which the appliance operation will be completed.

14. The household appliance of claim 13, wherein the transmitting of the activation signal is only in response to the time period remaining being less than or equal to a predefined time period.

15. The household appliance of claim 12, wherein the appliance operation is preparation of a food, and wherein the time at which the appliance operation will be completed is a time at which the food is prepared.

16. The household appliance of claim 12, wherein the appliance operation is cleaning of a floor, and the time threshold corresponds to a time to complete cleaning of the floor.

17. The household appliance of claim 12, wherein the household appliance is a robot vacuum cleaner.

18. The household appliance of claim 17, wherein the appliance operation is cleaning of a floor, and the time threshold corresponds to a time to complete cleaning of the floor
